(19) United States
(12) Patent Application Publication
(10) Pub. No.: US 2015/0366517 A1
(43) Pub. Date: Dec. 24, 2015

(54) METHOD AND MAGNETIC RESONANCE APPARATUS DEFINING A SEQUENCE PROTOCOL FOR THE MAGNETIC RESONANCE APPARATUS

(71) Applicant: Siemens Aktiengesellschaft, Muenchen (DE)

(72) Inventors: Uvo Hoelscher, Erlangen (DE); Dominik Paul, Bubenreuth (DE); Daniel Nico Splitthoff, Erlangen (DE)

(73) Assignee: Siemens Aktiengesellschaft, Muenchen (DE)

(21) Appl. No.: 14/747,362

(22) Filed: Jun. 23, 2015

(30) Foreign Application Priority Data
Jun. 23, 2014 (DE) 102014211958.8

(51) Int. Cl. A61B 5/00; A61B 5/055

(52) U.S. Cl. CPC A61B 5/7285; A61B 5/055

(57) ABSTRACT

In a method and magnetic resonance apparatus for defining a sequence protocol for the magnetic resonance apparatus, at least one parameter of the sequence protocol is determined, which is able to be changed automatically, and a provisional sequence protocol is then determined. MR data of an examination object are acquired, from which an overview image of the examination object is created. The provisional sequence protocol is adapted dependent on the overview image. A check is made as to whether the adapted sequence protocol exceeds a predefined stimulation threshold beyond which nerve stimulation of the examination object is too large. If the adapted sequence protocol exceeds the threshold, the at least one parameter is automatically changed such that the changed sequence protocol no longer exceeds the threshold. The changed sequence protocol corresponds to the sequence protocol to be determined.

### BACKGROUND OF THE INVENTION

1. Field of the Invention

The present invention concerns a method for defining a sequence protocol for a magnetic resonance apparatus, and a magnetic resonance apparatus designed to implement such a method.

2. Description of the Prior Art

Conventionally, a provisional magnetic resonance data acquisition sequence protocol is created, which is then adapted to the current situation (e.g. the position and alignment of the part of the patient's body to be examined). In such cases, the workflow is interrupted if nerve stimulation of the patient can occur as a result of the adapted MR protocol. During acquisition of data for MR images for answering orthopedic questions (so-called ortho protocols), the support of the patient and the anatomical alignment of the joint can produce widely fluctuating results with regard to nerve stimulation of the patient. Changes in relation to specific parameters of the MR protocol (e.g. FoV or timing parameters) can also lead to a threshold in respect of nerve stimulation being exceeded, so that the sequence protocol is not capable of achieving the intended measurement, and the measurement or the workflow must be interrupted in order to revise the sequence protocol.

In addition, the problem can arise in the prior art that the position and the alignment of the part of the patient's body to be examined (frequently also referred to as the positioning or angulation of the patient) leads, through the adaptation of the sequence protocol, to the maximum value of the magnetic field gradient performance (especially maximum rate of rise of one of the magnetic field gradients) being exceeded, which likewise then leads to an abort or to an interruption of the measurement in order to revise or adapt the sequence protocol. The maximum value of a magnetic field gradient performance can be exceeded because, in the creation of the provisional sequence protocol, the timing or time behavior is not calculated dependent on the orientation, and the worst case (which seldom occurs) is not taken into account.

### SUMMARY OF THE INVENTION

An object of the present invention is to perform the adaptation of the sequence protocol to the position and orientation of the part of the patient's body to be examined such that, if possible, no interruptions of the workflow or of the measurement occur.

In accordance with the present invention, a method is provided for defining a sequence protocol for a magnetic resonance system that includes the following steps. One or more parameters of the sequence protocol is/are defined, which can be changed automatically. Preferably only one parameter (e.g. the rise time of a magnetic field gradient) is defined in this step. A provisional sequence protocol is then defined. An overview image of the examination object is produced based on previously acquired MR data of the examination object. An automatic adaptation of the provisional sequence protocol is implemented depending on the overview image, wherein the provisional sequence protocol is adapted to the position and alignment (i.e. the angulation) of the volume segment to be examined (e.g. part of the body) of the examination object or patient. A check is made as to whether, during a measurement with the adapted sequence protocol, a predefined stimulation threshold is exceeded, beyond which a predefined (too large) nerve stimulation of the examination object occurs. If the adapted sequence protocol exceeds the predefined stimulation threshold, the parameter or the parameters is or are changed automatically so that the changed sequence protocol (i.e. the sequence protocol with the changed parameters) no longer exceeds the predefined stimulation threshold. The changed sequence protocol in such cases corresponds to the sequence protocol to be defined in accordance with the invention. If the adapted sequence protocol does not exceed the predefined stimulation threshold, the adapted stimulation protocol already corresponds to the sequence protocol to be defined.

The inventive method makes it possible for a protocol developer or medical specialist, when creating the provisional sequence protocol, to start from a normal or to some extent favorable position and orientation of the volume segment to be examined. This puts the protocol developer in the position of being able to create the provisional sequence protocol so that the measurement to be made with the protocol is executed in the shortest possible time and delivers the best possible results (informative MR images). For example with a turbo-echo-spin sequence a short echo spacing (ESP), i.e. spacing between two adjacent echoes, can be embodied in the planning. In many cases, the provisional sequence protocol can then be adapted to the current position and orientation of the volume segment to be examined without the predefined stimulation threshold being exceeded by this. If, however, this should actually be the case, the present invention ensures that the sequence protocol is also automatically changed in this case so that the changed sequence protocol no longer leads to the stimulation threshold being exceeded. Since the parameter or the parameters of the sequence protocol is or are automatically changed for this purpose, the workflow or the measurement advantageously no longer has to be interrupted for a manual input. It should be taken into account in such cases that the change of the parameter or parameters and thus the creation of the changed sequence protocol generally occurs all the more quickly the fewer parameters of the sequence protocol are proposed for automatic change. The automatic change can be carried out especially rapidly if only one parameter has been defined to be changed.

The step of defining the parameter or parameters can comprise the definition of a threshold beyond which the at least one parameter may not be changed and/or the definition of an associated range of values which defines a range in which the respective parameters may be changed. In such cases the threshold and/or the range of values is taken into account when automatically changing the at least one parameter so that the respective parameter does not exceed the threshold and/or lies within the range after the change has been made.

By defining the threshold and/or the range of values, the operator (e.g. the medical practitioner) can precisely define by how much the respective parameter may be changed. If the threshold and/or the range of values is predetermined by the protocol developer (which is generally the case) then it is to be assumed that the threshold and/or the range of values is predetermined so that the change of the parameter or parameters, taking into account the threshold and/or the range of values, also leads to the predefined stimulation threshold no longer being exceeded for a measurement with the changed sequence protocol.

The parameter or parameters which may be changed can be selected from a parameter group which comprises the following parameters: The dimensions of the field of view (FoV). The dimensions of the field of view are to be understood in such cases as the spatial size of the acquired volume segment, i.e. the surface to be acquired for each slice or also the volume of the volume segment to be acquired. The (maximum) rate of rise of the magnetic field gradient. As a rule the magnetic field gradients of a sequence protocol are switched with the same rate of rise. By changing this parameter both the problem of exceeding the stimulation threshold and also the problem of undershooting the magnetic field gradient performance (see below) can frequently be resolved. If only one parameter is determined for changing, this parameter is a favorite. The slice thickness. The resolution. The resolution is to be understood as the number of points which are acquired per slice or per volume segment. The read-out direction or the phase encoding direction(s). Two directions (for slice-by slice or two-dimensional acquisition) or three directions (for three-dimensional acquisition) are given by the volume segment to be examined. Through the parameters described here it is defined which of the given directions corresponds to the read-out direction. The remaining direction(s) then corresponds or correspond to the phase encoding direction(s). With three-dimensional data acquisition it can additionally be freely decided which of the two remaining directions corresponds to the first phasing coding direction, wherein the remaining third direction then corresponds to the second phase encoding direction. In such cases the three-dimensional volume segment can be scanned layer by layer, wherein the second phase encoding direction is at right angles to the slices.

For example, it can be predetermined in such cases that the FoV may be enlarged or reduced by a maximum of a predefined length or width or by a predefined percentage. The change of the (maximum) rate of rise of a magnetic field gradient can also be restricted in that it is predetermined for example that the (maximum) rate of rise may be increased or reduced at a maximum by a constant of the unit mT/ms/m or at a maximum by a predefined percentage. In a similar manner, it can be predetermined that the slice thickness may be increased (reduced) at a maximum by a predefined thickness or by a percentage.

In such cases, the at least one parameter can be changed so that the nerve stimulation caused by the changed sequence protocol lies very close to (but below) the predefined stimulation threshold. A further possibility is to only change the at least one parameter defined for change as little as possible by comparison with its original value, which generally also leads to the nerve stimulation caused by the changed sequence protocol lying very close to the predefined stimulation threshold.

Since the predetermined parameters of the sequence protocol are only changed if the sequence protocol adapted to the current position of the examination object exceeds the predefined stimulation threshold, the specification that the nerve stimulation caused by the changed sequence protocol lies very close to the predefined stimulation threshold advantageously leads to the parameters only having to be changed slightly as a rule. Since the change to the parameters mostly leads to a worsening of the results of the measurement (e.g. in respect of measurement time or quality of the MR images produced), this worsening can frequently be kept small by the above specification.

In the check as to whether the adapted sequence protocol exceeds the predefined stimulation threshold, it can also be checked whether the predetermined rate of rise of the magnetic field gradient is exceeded by the adapted sequence protocol. If this is the case, the at least one parameter is changed so that in the changed sequence protocol the predetermined rate of rise of the magnetic field gradient is no longer exceeded.

The simplest option for ensuring that the changed sequence protocol complies with the predetermined rate of rise of the magnetic field gradient is to adapt the predetermined rate of rise itself accordingly so that the correspondingly changed predetermined rate of rise is taken into account in the changed sequence protocol for the calculation of the gradients. The protocol developer can, for example, test in advance the extent to which the rate of rise is to be changed so that the sequence protocol can be used even in the worst case without the rate of rise predetermined for one of the three magnetic field gradients being exceeded. This ensures that the changed sequence protocol can be used for any position and/orientation of the part of the body to be examined, so that measurement is not aborted. Moreover, the rate of rise advantageously does not have to be set in advance in accordance with the worst case (which only seldom occurs) since, in accordance with the invention, the rate of rise is only be adapted automatically to the worst case if said case actually occurs.

The adaptation of the provisional sequence protocol depending on the overview image, i.e. the adaptation of the provisional sequence protocol to the position and alignment of the examination object, can include the following adaptations:
The setting of the orientation and position of the slices to be acquired or of the three-dimensional volume segment to be acquired is changed so that these slices or the volume segment path are or is adapted to the position and alignment of the part of the body to be examined or volume segment of the examination object. In the adaptation of the orientation and position of the slices the so-called axial plane and thus the normal of the plane can be greatly distorted in relation to the initial situation under which the provisional sequence protocol was created.
The adaptation of the dimensions of the field of view (FoV). The field of view is adapted to the dimensions of the volume segment of interest or to be examined of the examination object.
The number of slices to be acquired. The number of slices to be acquired is adapted to the dimension of the volume segment of interest of the examination object.
The setting or defining of which direction corresponds to the read-out direction along which a K space row is scanned and which direction corresponds to the phase encoding direction or directions.

In accordance with the present invention, a method is also provided for acquiring MR data of a predefined volume segment of an examination object with the aid of a magnetic resonance system. The inventive method includes defining a sequence protocol with an inventive method for defining a sequence protocol for a magnetic resonance system, as described above, and acquiring the MR data on the basis of the previously defined sequence protocol.

Since the sequence protocol is advantageously defined or adapted so that no interruption of the workflow is to be expected, the inventive method for acquiring the MR data can also be carried out advantageously without an interruption of the workflow.

The present invention also encompasses a magnetic resonance apparatus for acquisition of MR data of a volume segment within an examination object. This magnetic resonance apparatus has a scanner with a basic field magnet, a gradient field system, at least one RF antenna and a control computer for controlling the gradient field system and the at least one RF antenna, for receiving measurement signals picked up by the RF antenna or antennas, and for evaluating the measurement signals and for creating the MR images. The magnetic resonance apparatus is designed to acquire MR data of the examination object in order, on the basis of the MR data, to create an overview image of the examination object in order to adapt a provisional sequence protocol to the examination object depending on the overview image. The control computer is configured to check whether the adapted sequence protocol is exceeding a predefined stimulation threshold beyond which a predefined (i.e. too large) nerve stimulation of the examination object occurs. If the adapted sequence protocol exceeds the predefined stimulation threshold, the control computer is configured to change at least one parameter of the sequence protocol such that the changed sequence protocol no longer exceeds the predefined stimulation threshold, and to operate the scanner to acquire the MR data of the volume segment with the changed sequence protocol.

The advantages of the inventive magnetic resonance system essentially correspond to the advantages of the inventive method, as described above.

Furthermore the present invention also encompasses a non-transitory, computer-readable storage medium encoded with programming instructions, which can be loaded into a memory of a programmable controller or a processor of a magnetic resonance apparatus. The programming instructions cause all or various embodiments described above of the inventive method to be executed when by the controller or control computer of the magnetic resonance apparatus. The programming instructions may need program means, e.g. libraries and auxiliary functions, in order to achieve the corresponding forms of embodiment of the method. The programming instructions may be a source code (e.g. C++), which still has to be compiled (translated) and linked or that only has to be interpreted, or it can be executable software code that, for execution, only has to be loaded into the corresponding processing unit or control device.

The electronically readable data carrier can be, e.g. a DVD, a magnetic tape, a hard disk or a USB stick on which electronically-readable control information, is stored.

The present invention is especially suitable for acquiring MR images in which the sequence protocol is to be adapted to the position and/or an alignment of the volume segment to be acquired (i.e. of the patient). Naturally the present invention is not restricted to this preferred area of application, since the present invention can also be used for example if a sequence protocol is to be adapted in other ways.

### BRIEF DESCRIPTION OF THE DRAWINGS

FIG. 1 is a schematic illustration of an inventive magnetic resonance apparatus.

FIG. 2 is a flowchart of an embodiment of the inventive method for acquisition of MR data.

### DESCRIPTION OF THE PREFERRED EMBODIMENTS

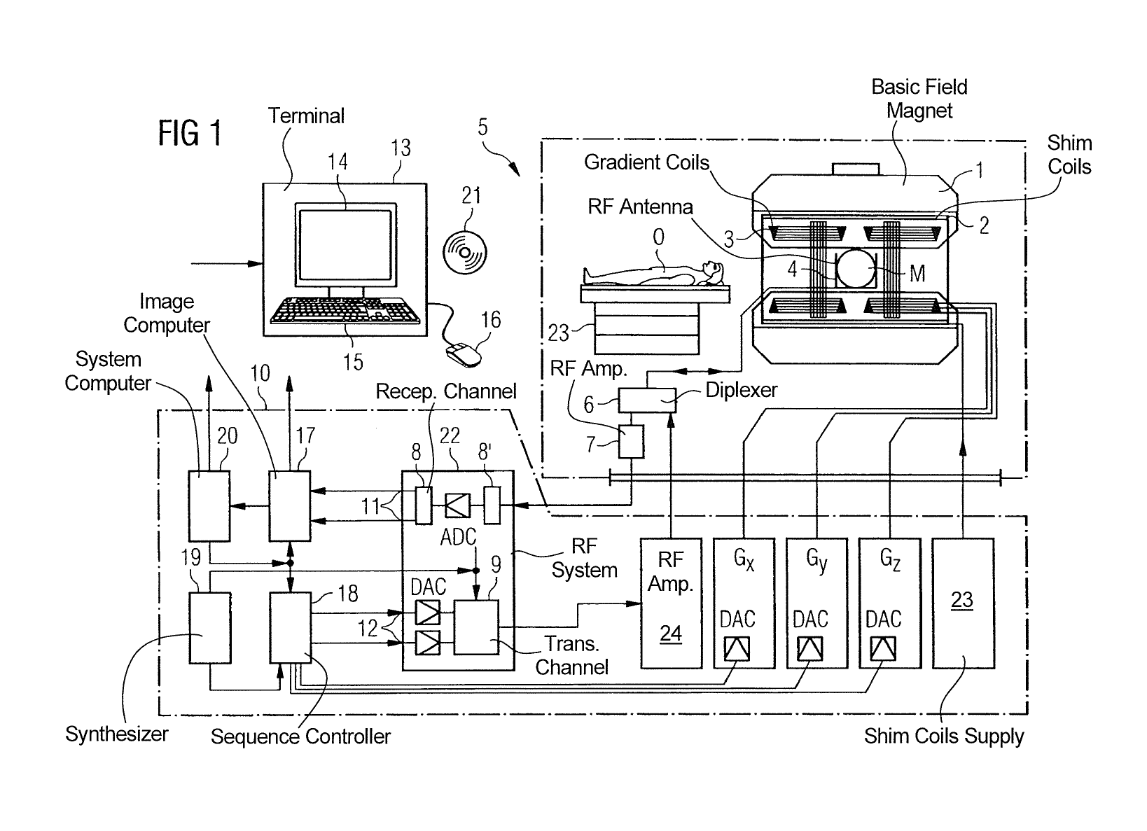

FIG. 1 is a schematic illustration of a magnetic resonance apparatus 5 (a magnetic resonance imaging or nuclear resonance tomography device). In this device a basic field magnet 1 creates a temporally constant strong magnetic field for polarization or alignment of the nuclear spin in an examination area of an object O, such as a part of the human body to be examined for example, which is continuously pushed, lying on a table 23, into the magnetic resonance system 5. The high homogeneity of the basic magnetic field required for nuclear magnetic resonance measurement is defined in a typically spherical measurement volume M, through which the parts of the human body to be examined are continuously pushed. To support the homogeneity demands and especially to eliminate temporally invariable influences, so-called shims made of ferromagnetic material are attached at a suitable point. Temporally variable influences are eliminated by shim coils 2, operated by a shim coils supply 23.

Implemented in the basic field magnet 1 is a cylindrical gradient field system 3, which is composed of three sub-windings. Each sub-winding is provided with power by an amplifier for creating a linear (also temporally changeable) gradient field in a respective direction of the Cartesian coordinate system. In this case the first sub-winding of the gradient field system 3 creates a gradient Gₓ in the x-direction, the second sub-winding creates a gradient G_{y} in the y-direction and the third sub-winding creates a gradient G_{z} in the z-direction. Each amplifier includes a digital-to-analog converter, which is controlled by a sequence controller 18 for creation of gradient pulses at the correct time.

Within the gradient field system 3 are one (or more) radio frequency antennas 4, which convert the radio frequency pulses emitted by a radio-frequency power amplifier 24 into a magnetic alternating field for exciting the nuclei and aligning the nuclear spin of the object O to be examined or of the area of the object O to be examined. Each radio-frequency antenna 4 consists of one or more RF transmit coils and one or more RF receive coils in the form of an annular, preferably linear or matrix-type arrangement of component coils. The alternating field emanating from the preceding nuclear spin, i.e. usually the nuclear spin echo signals caused by a pulse sequence of one or more radio frequency pulses and one or more gradient pulses is also converted by the RF receive coils of the respective radio-frequency the antenna 4 into a voltage (measurement signal) which is fed via an amplifier 7 to a radio-frequency receive channel 8 of the radio-frequency system 22. The radio-frequency system 22, which is part of a control device 10 of the magnetic resonance apparatus 5, also includes a transmit channel 9 in which the radio frequency pulses for the excitation of the magnetic nuclear resonance are created. In such cases the respective radio-frequency pulses, as a result of a pulse sequence predetermined by the system computer 20, are represented in the sequence controller 18 digitally as a sequence of complex numbers. This number sequence is supplied as a real part and an imaginary part via respective inputs 12 to a digital-to-analog converter in the radio-frequency system 22 and is supplied by the converter to a transmit channel 9. In the transmit channel 9 the pulse sequences are modulated up to a radio-frequency carrier signal of which the basic frequency corresponds to the resonant frequency of the nuclear spins in the measurement volume.

The components within the dot-dash outline 5a are commonly called a scanner.

The switchover from transmit to receive mode is undertaken via a transmit-receive switch 6. The RF transmit coils of the radio-frequency antenna(s) 4 emit the radio frequency pulses for exciting the nuclear spin into the measurement volume M and resulting echo signals are sampled via the RF receive coil(s). The correspondingly obtained nuclear resonance signals are demodulated in the receive channel 8′ (first demodulator) of the radio-frequency system 22 phase-sensitively to an intermediate frequency, digitized in the analog-to-digital converter (ADC) and output via the output 11. This signal is further demodulated to the frequency 0. The demodulation to the frequency 0 and the separation into real and imaginary part takes place after the digitization in the digital domain in a second demodulator 8. From the measurement data obtained in this way via an output 11 an MR image is reconstructed by an image processor 17. The administration of the measurement data, the image data and the control programs is done by the system computer 20. On the basis of a specification with control programs, the sequence controller 18 checks the creation of the respective desired pulse sequences and the corresponding scanning of the k-space. In particular the sequence controller 18 in such cases controls the correctly timed switching of the gradients, the radiation of the radio frequency pulses with defined phase amplitude and the receipt of the nuclear resonance signals. The time base for the radio-frequency system 22 and the sequence controller 18 is made available by a synthesizer 19. The corresponding control programs for creating an MR image, which are stored on a DVD 21 for example, are selected and the created MR image is presented via a computer terminal 13, which includes a keyboard 15, a mouse 16 and a display screen 14.

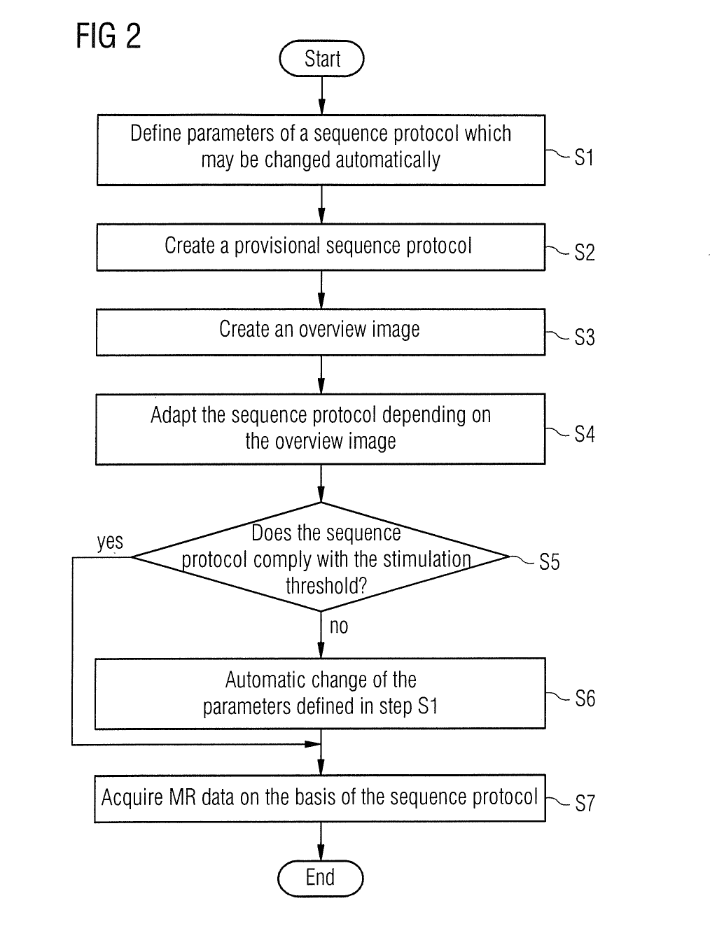

FIG. 2 is a flowchart of an inventive method for acquisition of MR data with a magnetic resonance system.

In step S1 those parameters of the sequence protocol which may be changed automatically (i.e. without user inputs) are determined. In addition to the parameters an associated range of values can be defined for each parameter, wherein the respective range of values defines those values of the respective parameter which may be used for an automatic change of the respective parameter.

In step S2 a provisional sequence protocol is created by the protocol developer (as a rule a medical practitioner) in order to acquire MR data in a predefined volume segment (part of the body to be examined) of a patient. In step S3 an overview image of the patient is created, which especially contains the predefined volume segment. In step S4 the provisional sequence protocol is adapted depending on the overview image such that a predefined volume segment can be acquired in an optimal manner in the current position and alignment which are to be taken from the overview image. For example, in this step the position and the alignment of the slices to be acquired are adapted to the position and alignment of the volume segment.

In step S5 a check is made as to whether, in a measurement with the adapted sequence protocol, the stimulation threshold of a nerve stimulation of the patient would be complied with. In this step S5 a check can additionally be made as to whether a predetermined (maximum) rate of rise of each of the three magnetic field gradients Gx, Gy, Gz would not be exceeded in a measurement with the adapted sequence protocol. If this is the case (i.e. in a measurement with the adapted sequence protocol neither the stimulation threshold nor the predetermined rates of rise of the three magnetic field gradients Gx, Gy, Gz would be exceeded), in step S7 the MR data is acquired on the basis of the adapted sequence protocol.

If, however, it is recognized during the checking that either the stimulation threshold will be exceeded or, at least for one of the magnetic field gradients, the predetermined rate of rise will be exceeded, in step S6 the parameters defined in step S1 or also S4 are automatically changed such that, in the measurement with the changed sequence protocol, neither the stimulation threshold nor the predetermined rates of rise of the magnetic field gradients Gx, Gy, Gz are exceeded. Subsequently, in step S7, the MR data is acquired with the use of the changed sequence protocol.

Although modifications and changes may be suggested by those skilled in the art, it is the intention of the inventor to embody within the patent warranted hereon all changes and modifications as reasonably and properly come within the scope of his contribution to the art.

We claim as our invention:

1. A method for defining a sequence protocol for a magnetic resonance apparatus, said method comprising:
in a processor provided with a sequence protocol, comprising a plurality of parameters, for operating a magnetic resonance scanner of a magnetic resonance apparatus, determining at least one of said parameters that is able to be changed automatically;
in said processor, determining a provisional sequence protocol based on said sequence protocol;
from said processor, operating the magnetic resonance scanner, while an examination object is situated therein, according to said provisional sequence protocol to acquire magnetic resonance data from the examination object that represent an overview image of the examination object;
in said processor, automatically adapting said provisional sequence protocol dependent on said overview image, thereby producing an adapted sequence protocol;
in said processor, automatically checking whether said adapted sequence protocol exceeds a predetermined stimulation threshold that defines a permissible level of nerve stimulation of the examination object while magnetic resonance data are being acquired from the examination object;
in said processor, if the adapted sequence protocol exceeds said predetermined stimulation threshold, automatically changing said at least one parameter to produce a changed sequence protocol that does not exceed said predetermined stimulation protocol but that otherwise corresponds to said sequence protocol; and
making said changed protocol available at an output of the processor in an electronic form with which said magnetic resonance scanner is operable.

2. A method as claimed in claim 1 comprising determining said at least one parameter that can be changed automatically by defining a change threshold beyond which said at least one parameter may not be changed, and automatically changing said at least one parameter dependent on said change threshold.

3. A method as claimed in claim 1 comprising selecting said at least one parameter from the group of parameters consisting of dimensions of a field of view with which magnetic resonance data are to be acquired with said sequence protocol, a maximum rate of rise of a magnetic field gradient generated in said magnetic resonance scanner by said sequence protocol, a slice thickness within the examination object from which magnetic resonance data are to be acquired, a resolution of said magnetic resonance data, a read-out direction produced by a gradient magnetic field in said magnetic resonance scanner, and a phase coding direction produced by a gradient field in said magnetic resonance scanner.

4. A method as claimed in claim 1 comprising changing said at least one parameter to cause nerve stimulation in the examination object produced by said sequence protocol to be as close as possible to, but not to exceed, said predetermined stimulation threshold.

5. A method as claimed in claim 1 comprising:
in said processor, also checking whether a rate of rise of a magnetic field gradient produced in said magnetic resonance scanner by said adapted sequence protocol exceeds a maximum rate of rise; and
if said rate of rise exceeds said maximum rate of rise, changing said at least one parameter to make said rate of rise in said changed sequence protocol not exceed said maximum rate of rise.

6. A method as claimed in claim 1 comprising adapting said provisional sequence protocol by at least one of setting an orientation of a slice of the examination object from which magnetic resonance data are to be acquired, setting dimensions of a field of view within said magnetic resonance scanner produced by said adapted sequence protocol, setting a number of slices within the examination object from which magnetic resonance data are to be acquired, setting a read-out direction produced by a gradient magnetic field in said magnetic resonance scanner, and setting a phase coding direction produced by a gradient field in said magnetic resonance scanner.

7. A method as claimed in claim 1 comprising, from said processor, operating said magnetic resonance scanner according to said changed sequence protocol to acquire magnetic resonance data from the examination object.

8. A magnetic resonance apparatus comprising:
a magnetic resonance scanner;
a processor provided with a sequence protocol, comprising a plurality of parameters, for operating a magnetic resonance scanner of a magnetic resonance apparatus, said processor being configured to determine at least one of said parameters that is able to be changed automatically;
said processor being configured to determine a provisional sequence protocol based on said sequence protocol;
said processor being configured to operate the magnetic resonance scanner, while an examination object is situated therein, according to said provisional sequence protocol to acquire magnetic resonance data from the examination object that represent an overview image of the examination object;
said processor being configured to automatically adapt said provisional sequence protocol dependent on said overview image, thereby producing an adapted sequence protocol;
said processor being configured to automatically check whether said adapted sequence protocol exceeds a predetermined stimulation threshold that defines a permissible level of nerve stimulation of the examination object while magnetic resonance data are being acquired from the examination object;
said processor, if the adapted sequence protocol exceeds said predetermined stimulation threshold, being configured to automatically change said at least one parameter to produce a changed sequence protocol that does not exceed said predetermined stimulation protocol but that otherwise corresponds to said sequence protocol; and
said processor being configured to make said changed protocol available at an output of the processor in an electronic form with which said magnetic resonance scanner is operable.

9. A non-transitory, computer-readable data storage medium encoded with programming instructions, said storage medium being loaded into a control computer of a magnetic resonance apparatus, that also comprises a magnetic resonance scanner, and said programming instructions causing said control computer to:
receive a sequence protocol, comprising a plurality of parameters, for operating a magnetic resonance scanner of a magnetic resonance apparatus, and determine at least one of said parameters that is able to be changed automatically;
determine a provisional sequence protocol based on said sequence protocol;
operate the magnetic resonance scanner, while an examination object is situated therein, according to said provisional sequence protocol to acquire magnetic resonance data from the examination object that represent an overview image of the examination object;
adapt said provisional sequence protocol dependent on said overview image, thereby producing an adapted sequence protocol;
check whether said adapted sequence protocol exceeds a predetermined stimulation threshold that defines a permissible level of nerve stimulation of the examination object while magnetic resonance data are being acquired from the examination object;
if the adapted sequence protocol exceeds said predetermined stimulation threshold, automatically change said at least one parameter to produce a changed sequence protocol that does not exceed said predetermined stimulation protocol but that otherwise corresponds to said sequence protocol; and
make said changed protocol available at an output of the control computer in an electronic form with which said magnetic resonance scanner is operable.
